(19) United States
(12) Patent Application Publication
(10) Pub. No.: US 2020/0188316 A1
(43) Pub. Date: Jun. 18, 2020

(54) TRANSDERMAL FORMULATIONS SUITABLE FOR ADMINISTRATION OF NATURAL PRODUCTS COMPRISING PLANT FLOUR AND AN ADHESIVE

(71) Applicants: Robert BENDER, Ottawa (CA); ZEPHYRIOUS HEALTH, INC., OTTAWA (CA)

(72) Inventor: ROBERT BENDER, OTTAWA (CA)

(21) Appl. No.: 16/640,913

(22) PCT Filed: Aug. 24, 2018
(86) PCT No.: PCT/IB18/56467

§ 371 Date: Feb. 21, 2020

Related U.S. Application Data
(60) Provisional application No. 62/549,455, filed on Aug. 24, 2017.

(51) Int. Cl. A61K 9/70; A61K 36/185; A61K 36/9066; A61K 31/352; A61K 31/05

(52) U.S. Cl. CPC A61K 9/7053; A61K 36/185; A61K 31/05; A61K 31/352; A61K 36/9066

(57) ABSTRACT

Pharmaceutical and nutraceutical compositions suitable for transdermal administration of plant-derived medicinal compounds are disclosed. The compositions comprise ground plant material (plant flour) and a dermally acceptable adhesive. In preferred embodiments, the plant flour is derived from Cannabis sp. and the adhesive is a polymer selected from the group consisting of acrylic polymers, silicone polymers, urethanes, isobutylenes, polyisobutylenes, vinyl acetate, and styrene polymers or copolymers.

### RELATED APPLICATIONS

This application claims priority from U.S. Provisional Patent Application No. 62/549,455, filed on Aug. 24, 2017, which is incorporated herein by reference in its entirety.

### FIELD

This disclosure relates to preparations and formulations for transdermal delivery and in particular, direct formulations for transdermal delivery of natural products.

### BACKGROUND

Natural products for medicinal or recreational use are commonly ingested or inhaled. Where the ‘active’ ingredients meet well understood physical conditions, it is common to prepare extracts or concentrates for convenience of administration and standardization. Certain pharmacologically active materials such as cannabinoids and terpenoids have solubility properties which make it possible for them to be absorbed transdermally.

It is well known art to prepare formulations of such materials for transdermal administration by extracting the active materials from the naturally occurring whole plant and to then combining the active components with solvents and adhesives to create thereby devices which can continuously present the active components to the skin. Absorption through the skin is generally via passive diffusion and, accordingly, the rate of dosing is proportional to the area of the device and the (measured) flux rate which in turn depends on the concentration of the material to be delivered.

In transdermal delivery devices, the active component is generally dissolved in a vehicle which allows uniform presentation of the substance to the skin (intimate contact and mobility) which is achieved by using a viscous solvent or adhesive. A reservoir is created so that excess material is continuously presented to the skin and various barrier membranes may be used to created/control concentration gradients. In the simplest of such devices, the concentration of the active material gradually lowers as the material moves from the device through the skin; equilibrium is not achieved unless the subcutaneous tissue becomes saturated with the active. As the concentration lowers, the rate of delivery of the active materials decreases.

If a uniform rate of delivery is desired, it is therefore desirable to present a ‘saturated-plus-excess’ composition to the skin so that as the active enters the body the concentration of active in the adhesive proximate to the skin remains constant (as the excess dissolves into the carrier). This type of device has the advantage of maintaining a constant rate of delivery until the active is close to exhausted and also can be formulated to use less active material depending on the solubility properties of the active.

Both the simple reservoir and the saturated-plus-excess designs are in current use for the delivery of drugs. The advantages of saturated-plus-excess designs include better uniformity with respect to delivery rates and more efficient use of material since the diffusion gradient is maintained by the excess of active compound over saturation levels.

It is general practice to prepare transdermal delivery devices through either film casting or extrusion and from chemically well-characterized extracts of natural materials.

### SUMMARY

An object of the present disclosure is to provide direct formulations for transdermal delivery of natural products. In accordance with an aspect of the present disclosure, there is provided a pharmaceutical or nutraceutical composition comprising an adhesive suitable for application to the skin and finely ground plant material.

In accordance with another aspect of the disclosure, there is provided a composition comprising cannabis flour and an adhesive suitable for application to the skin.

In accordance with another aspect of the disclosure, there is provided a method of manufacturing a natural product composition, the method comprising mechanically mixing cannabis flour in an adhesive suitable for application to the skin.

In accordance with another aspect of the disclosure, there is provided a kit comprising a composition comprising cannabis flour and an adhesive suitable for application to the skin and a pad.

The disclosure provides a pharmaceutical or nutraceutical composition comprising a plant flour mixed with an adhesive. In one embodiment, the plant flour comprises one or more plants selected from the group consisting of Cannabis, Curcurma (turmeric), Olea (olive), Theobroma (cocoa), Camellia (tea), Vitis (grape) and Cinnamomum (cinnamon).

In another embodiment, the plant flour has an average particle diameter of 30 μm-80 μm. In another embodiment, the plant flour has a particle diameter that passes through a screen of 80 mesh-400 mesh. In another embodiment, the plant flour is mixed with the adhesive at a ratio of plant flour to adhesive from 1:1 (w/w) to 1:100 (w/w).

In yet another embodiment, the composition has a viscosity of between 4-1000 cP. In another embodiment, the adhesive is selected from the group consisting of an acrylic polymer, a silicone polymer, urethane, isobutylene, polyisobutylene, vinyl acetate and styrene polymers or copolymers.

In another embodiment, the plant flour comprises cannabis. In one aspect of this embodiment, the adhesive is acrylic polymer, silicone polymer or polyisobutylene (PIB).

The disclosure also provides a method of manufacturing a pharmaceutical or nutraceutical composition comprising grinding plant material into a flour and mixing the flour with an adhesive. In one embodiment, the plant flour comprises one or more plants selected from the group consisting of Cannabis, Curcurma (turmeric), Olea (olive), Theobroma (cocoa), Camellia (tea), Vitis (grape) and Cinnamomum (cinnamon).

In another embodiment, the plant flour has an average particle diameter of 30 μm-80 μm. In another embodiment, the plant flour has a particle diameter that passes through a screen of 80 mesh-400 mesh. In another embodiment, the plant flour is mixed with the adhesive at a ratio of plant flour to adhesive from 1:1 (w/w) to 1:100 (w/w).

In yet another embodiment, the composition has a viscosity of between 4-1000 CPoise. In another embodiment, the adhesive is selected from the group consisting of an acrylic polymer, a silicone polymer, urethane, isobutylene, PIB, vinyl acetate and styrene polymers or copolymers.

In another embodiment, the plant flour comprises cannabis. In one aspect of this embodiment, the adhesive is acrylic polymer, silicone polymer or PIB.

The disclosure also provides a method of administering tetrahydrocannabinol (THC), cannabidiol (CBD) and/or terpenes to a subject in need thereof comprising mixing a cannabis flour with an adhesive. In one embodiment, the plant flour has an average particle diameter of 30 μm-80 μm. In another embodiment, the plant flour has a particle diameter that passes through a screen of 80 mesh-400 mesh. In another embodiment, the plant flour is mixed with the adhesive at a ratio of plant flour to adhesive from 1:1 (w/w) to 1:100 (w/w).

In another embodiment, the composition has a viscosity of between 4-1000 CPoise. In another embodiment the indicated adhesive is selected from an acrylic polymer, a silicone polymer, urethane, isobutylene, PIB, vinyl acetate and styrene polymers or copolymers. In another embodiment, the adhesive is acrylic polymer, silicone polymer or PIB.

The disclosure also provides a kit comprising a composition comprising plant flour and an adhesive, wherein the plant flour is mixed with the adhesive at a ratio of plant flour to adhesive from 1:1 (w/w) to 1:100 (w/w), wherein the adhesive is cast with a dry thickness of between about 45 μm and about 95 μm, and wherein the adhesive is laminated with a backing membrane. In one embodiment, the plant flour comprises one or more plants selected from the group consisting of Cannabis, Curcurma (turmeric), Olea (olive), Theobroma (cocoa), Camellia (tea), Vitis (grape) and Cinnamomum (cinnamon).

In another embodiment, the plant flour has an average particle diameter of 30 μm-80 μm. In another embodiment, the plant flour has a particle diameter that passes through a screen of 80 mesh-400 mesh. In another embodiment, the indicated adhesive is selected from the group consisting of an acrylic polymer, a silicone polymer, urethane, isobutylene, polyisobutylene, vinyl acetate and styrene polymers or copolymers.

In another embodiment, the plant flour comprises cannabis. In one aspect of this embodiment, the adhesive is acrylic polymer, silicone polymer or PIB.

In another embodiment, the backing membrane is constructed of a material selected from the group consisting of polyesters, polycarbonates, polyimides, polyethylene, poly(ethylene terphthalate), polypropylene, polyurethanes and polyvinylchlorides.

### DETAILED DESCRIPTION

The present disclosure provides methods to formulate a presentation adhesive directly from finely ground plant material by mechanical mixing with an adhesive composition in which the pharmacologically active components are sparingly soluble. This mixture then becomes a dispersion of particulate material from which some of the active materials are extracted by the adhesive itself acting as the solvent. The adhesive may itself comprise a polymer in a solution of a low molecular weight organic solvent or may be a polymer of appropriate viscosity and properties which make it capable of dissolving the active agent(s). The adhesive mixture must be appropriate for use in contact with the skin (e.g. non-irritant).

If the adhesive is, for example, an acrylic or silicone polymer, it may be formulated with a volatile solvent or mixture of solvents to facilitate fabrication of devices in processes where the lower molecular weight components may be removed by (for example) evaporation. Alternately, for film extrusion processes the only condition that need be met is that the active components be (sparingly) soluble in the adhesive. Small amounts of medium molecular weight aliphatic solvents (oils) may be used to modify both solubility of actives and mechanical properties of the reservoir.

As an example of a simple reservoir transdermal delivery device, a quantity of a specific strain of cannabis may be dried and finely milled to a uniform flour and mixed with an adhesive. Note that this process requires care to ensure uniformity of the flour with respect to special distribution of active components. In certain embodiments, a uniform flour has at least 50, 60, 70, 75, 80, 85, 90, 95 or 99% of the particles in the flour is within 25% of the size of the average sized particle Typically the composition of commercial cannabis is in the neighborhood of 20% total actives (THC, CBD and terpenes). Based on analytical data, the proportion of dried milled material to an adhesive formulation may be calculated and after mixing to uniformity the adhesive+active+inert mixture may be incorporated into transdermal delivery devices. The manufacturing process parameters need to be set to deliver an appropriate amount for the intended purpose of the delivery device. Specifically, the area of the device can be set to determine total transdermal flux and the amount of reservoir material will determine the duration during which the intended flux rate can be maintained. In a simple reservoir device the concentration of the active will drop with time and thus the flux rate will decrease with time. It is usual and customary in the field to design for excess chemical drive vs the limit set by the flux rate and then allow such devices to gradually deplete; a practical consequence is that in use such devices deliver approximately half of their contents. This is wasteful (unnecessarily costly) and potentially a source of material for diversion.

If the adhesive is chosen to have low solubility for the active components, a saturation plus excess situation can be achieved with the advantages of requiring lower quantities of active material to maintain chemical drive (flux rate) and additionally leaving less material in the device at the end of its predicted life.

Note that compound reservoirs (membrane separated) may be used to control delivery of actives into the skin and that such designs can equally be made with solid-in-polymer solvents or solid-in-gel systems. The basic novelty of the approach described herein is the use of unaltered natural products containing a number of active components which can be dispersed as a powder into a polymer or polymer-solvent system which acts simultaneously as an extraction medium and reservoir of active materials for presentation to the skin. This novelty has utility directed towards controlled administration of cannabinoids which occur as complex natural mixtures. Utility for administration of other natural products such as codeine or certain hormones and steroid analogs is also anticipated.

In certain embodiments, the transdermal delivery devices described herein are prepared by casting a wet pharmaceutical formulation layer as described herein at a known thickness onto a suitable release liner. In its simplest form, the pharmaceutical formulation may comprise plant flour mixed with a dermatologically-acceptable adhesive. The pharmaceutical formulation may additionally comprise one or more additional excipients, including a carrier oil, penetration enhancers and hydrophilic materials. Typically, the pharmaceutical formulation are cast at a wet thickness of between about 240 μm to about 550 μm, to provide a dry thickness of between about 45 μm and about 95 μm, suitably between about 80 μm and about 85 μm. After casting, the layer is dried, and then laminated with a backing membrane. A suitable container or closure system may be used protect the transdermal patch during transportation and storage.

In certain embodiments, the adhesive is an acrylic or silicone polymer. In other embodiments, the adhesive is selected from acrylate, synthetic rubber, silicone, polyurethane, polyisobutylene, polyvinyl acetate and/or polystyrene. In certain embodiments, the adhesive is selected for its low solubility for the active components of the plant flour that the adhesive is mixed with. For example, cannabis typically contains in the neighborhood of 20% total actives (THC, CBD and terpenes). In certain embodiments, an adhesive that has limited solubility (i.e. sparingly soluble) to these actives is chosen. In specific embodiments, the adhesive is an acrylic or silicone polymer. In one embodiment, the adhesive is polyisobutylene.

A combination of plant flour and adhesive, wherein the adhesive has a limited solubility of the actives in the plant flour can be attained by using a greater amount of plant flour. The amount of plant flour used depends on the natures of the active ingredients in the flour and the solubility of those ingredients in the adhesive.

In certain embodiments, an adhesive has limited solubility for a substance when less than 1 g of the substance can be solubilized in 10 g of the adhesive. In other embodiments, an adhesive has limited solubility for a substance when less than 0.5, 0.1, 0.05, 0.01, 0.001, 0.0005, 0.0001 or 0.00001 g of the substance can be solubilized in 10 g of the adhesive.

Suitable backing membranes may be occlusive or non-occlusive. Where a non-occlusive backing membrane is used, it is desirable to use a fully occlusive container or closure system to prevent degradation of the cast pharmaceutical formulation layer prior to use. The backing membrane may be of any thickness, but is suitably between about 10 to 260 μm thick. Suitable materials include, but are not limited to, synthetic polymers including, for example, polyesters, polycarbonates, polyimides, polyethylene, poly(ethylene terphthalate), polypropylene, polyurethanes and polyvinylchlorides. The backing membrane may also be a laminate comprising additional layers that may include vapor deposited metal, such as aluminum, additional synthetic polymers, and other materials, to enable a heat seal, such as EVA copolymer.

The release liner is typically disposed on an opposite surface of the pharmaceutical formulation layer to the backing membrane and provides a removable protective or impermeable layer, usually but not necessarily rendered non-stick so as to not adhere to the pharmaceutical formulation layer. The release liner serves to protect the pharmaceutical formulation layer during storage and transit, and is intended to be removed during use. The release liner may be formed from the same materials used for the backing membrane, but may be formed from metal foils, Mylar®, polyethylene terephthalate, siliconized polyester, fumed silica in silicone rubber, polytretrafluoroethylene, cellophane, siliconized paper, aluminized paper, polyvinyl chloride film, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene, and styrene-isoprene copolymers, polyethylene, and polypropylene.

In certain embodiments, the release liner is an occlusive or semi-occlusive backing film being compatible with the pharmaceutically-acceptable adhesive present in the pharmaceutical formulation layer.

Suitable release liners made by other manufacturers may also be used. The release liner may be of any thickness known in the art. Suitably the release liner has a thickness of about 0.01 mm to about 2 mm.

In certain embodiments, the container or closure system may be made from a range of materials suitable for protecting the packaged transdermal patch from moisture and light.

According to certain embodiments, the compositions described herein also include a penetration enhancer. Penetration enhancers serve to promote the percutaneous absorption of substances by temporarily diminishing the impermeability of the skin. Importantly, when included in the compositions described herein, the penetration enhancer must not compromise the release characteristics of the adhesive.

In certain embodiments, the penetration enhancer and the quantities in which it is added should be nontoxic, non-irritating, non-allergenic, odorless, tasteless, colorless, soluble, and compatible with the plant flour included in the compositions described herein. Importantly, the enhancer should not lead to the loss of bodily fluids, electrolytes and other endogenous materials, and skin should immediately regain its barrier properties on its removal. Examples of penetration enhancers suitable for inclusion into the pharmaceutical formulation described herein include, but are not limited to, sugar fatty acid esters and ethers, C8-C18 fatty alcohol, azone, oleic ethers, terpenes and ethoxy ethanols. In some embodiments, when used, the penetration enhancer is present in the pharmaceutical formulation at a concentration of between about 1.4% (w/w) and about 15% (w/w). In other embodiments, the penetration enhancer is selected from polyoxyethylene oleyl ether, obtainable under the trade name Brij 93®, or 2-(2-ethoxyethoxy)ethanol, obtainable under the trade name Transcutol®, or menthol.

In certain embodiments, the transdermal device comprises at least one reservoir and a membrane that controls the release of drug to the skin. In certain embodiments, the membrane is a microporous membrane. The microporous membrane may have pores with diameters in the range of about 0.05 to about 10 μm, or more specifically in the range of about 0.1 to about 6.0 μm.

### Definitions

The term “plant flour” refers to milled and/or dried powder derived from any part of the plant. As will be evident to a person of skill in the art, the production of plant flour may be accomplished by a variety of methods as known in the art. Further, such methods may employ one or more devices or apparatuses known in the art, including, but not limited to a Wiley mill, Thomas mill, Cyclotech mill, jet mill, centrifugal mill, pin mill with or without air classification, or any combination thereof.

In one embodiment, the plant flour is made up of substantially only one type of plant or one structural and/or physiological unit of a plant type. In another embodiment, the plant flour is made up of only one type of plant or one structural and/or physiological unit of a plant type. In other embodiments, the flour is made up of two or more plant types or two or more structural and/or physiological units of a plant type.

According to certain embodiments, the flour comprises one or more materials soluble in non-aqueous (organic) solvent. In certain embodiments, the materials are sparingly soluble. The materials can be pharmaceutically active ingredients of the flour. For example, flour derived from cannabis can contain THC, CBD and terpenes. In other embodiments, only one active is present. For example, flour derived from turmeric comprises curcumin.

It is also contemplated that the flour may be further processed to comprise one or more additional characteristics. For example, the flour may be defatted with chloroform or other appropriate solvent, dried to a particular moisture content or range, or the flour may be sifted or sized by passing through a mesh screen or the like to obtain compositions having a desired particle size, size range or size distribution. It is also contemplated that seeds employed to produce flour may be subject to one or more pretreatments, for example, but not limited to dehulling, fermenting or both. Other pretreatments or processing conditions, for example, but not limited to dry processing or wet processing as would be known by a person of skill in the art also may be employed in the method described herein.

Without wishing to be limiting, flour can pass through a screen comprising a filter size of between about 40 mesh to about 600 mesh or more, for example, 40, 50, 60, 70, 80, 90, 100, 110, 120, 130, 140, 150, 160, 170, 180, 190, 200 mesh or any value therein between. In certain embodiments, the flour can pass through a screen with a filter size from 80 mesh to 400 mesh, from 100 mesh to 350 mesh, from 150 mesh to 300 mesh, or from 180 mesh to 200 mesh. Further, the flour may be characterized as passing through a mesh size defined by any two of the values listed above. In some embodiments the flour may also be characterized as flour with average particle sizes of about 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, or 190 μm. In certain embodiments, the average particle size of the flour is from 10 μm to 200 μm, from 20 μm to 180 μm, from 30 μm to 170 μm, from 40 μm to 160 μm, from 50 μm to 150 μm, from 60 μm to 140 μm, from 70 μm to 130 μm from 80 μm to 120 μm or from 90 μm to 110 μm.

Plants that can be used according to this disclosure include but are not limited to the genera of plants selected from Aconitum, Aloe, Scutellaria, Arnica, Panax, Ocimum, Vaccinium, Areca, Aristolochia, Citrus, Juglans, Actaea, Vachellia, Uncaria, Chelidonium, Ranunculus, Asteraceae, Larrea, Cinnamomum, Syzygium, Erythroxylon, Tussilago, Symphytum, Sida, Taraxacum, Harpagophytum, Digitalis, Datura, Angelica, Echinacea, Eleutherococcus, Ephedra, Aristolochia, Oenothera, Euphrasia, Foeniculum, Tanacetum, Allium, Matricaria, Teucrium, Ginkgo, Panax, Hydrastis, Paradisi, Graviola, Camellia, Senecio, Crataegus, Hyoscyamus, Epimedium, Aesculus, Equisetum, Callilepsis, Datura, Lobelia, Glycyrrhiza, Convallaria, Lobelia, Rubia, Ephedra, Cannabis, Ilex, Silybum, Viscum, Artemisia, Aristolochia, Azadirachta, Origanum, Nerium, Papaver, Origanum, Petroselinum, Mentha, Trifolium, Chamaemelum, Rosmarinus, Salvia, Sassafras, Serenoa, Senna, Eleutherococcus, Annona, Spinacia, Drimia, Stevia, Hypericum, Melaleuca, Cucurma, Nicotiana, Valeriana, Dionaea, Salix, Gaultheria, Isatis and Artemisia.

The plant flour can be made up of any one or more structural and/or physiological units of a plant. The structural and/or physiological unit may be an organized unit such as, for example, a plant tissue, or a plant organ differentiated into a structure that is present at any stage of a plant's development. Such structures include but are not limited to fruits, shoots, stems, leaves, flowers, petals, and roots. Structural and/or physiological units also include whole plants, shoot vegetative organs/structures (e.g. leaves, stems and tubers), roots, flowers and floral organs/structures (e.g. bracts, sepals, petals, stamens, carpels, anthers and ovules), seeds (including embryo, endosperm, and seed coat) and fruits (the mature ovary), plant tissues (e.g. vascular tissue, ground tissue, and the like) and cells (e.g. guard cells, egg cells, trichomes and the like), and progeny of same.

The term “adhesive” refers to a substance that may be integrated with the plant flour to hold the structure in contact with a subject's skin. The adhesive should be compatible with the plant flour and be a non-irritant. In certain embodiments, the adhesive should be able to solubilize the active ingredients of the plant flour, at least sparingly, and facilitate the entry of the active ingredients into the user's bloodstream. In some embodiments, the adhesive material is comprised of a synthetic material including but not limited to acrylate, synthetic rubber, silicone, polyurethane, polyisobutylene, polyvinyl acetate and/or polystyrene, etc. The adhesive strips can be cast directly onto the skin-facing side of the backing layer or a rate controlling membrane.

According to the present disclosure, the adhesive can be mixed with the plant flour at a mass ratio of plant flour to adhesive of between 1:1 (w/w) to 1:100 (w/w). In other embodiments, the ratio is between 1:2 (w/w) and 1:50 (w/w), 1:3 (w/w) and 1:40 (w/w), 1:4 (w/w) and 1:30 (w/w). 1:5 (w/w) and 1:20 (w/w) or 1:7 (w/w) and 1:10 (w/w). In other embodiments, the ratio is about 1:1 (w/w), 1:2 (w/w), 1:3 (w/w), 1:4 (w/w), 1:5 (w/w), 1:5 (w/w), 1:7 (w/w), 1:8 (w/w), 1:9 (w/w), 1:10 (w/w), 1:15 (w/w), 1:20 (w/w), 1:30 (w/w), 1:40 (w/w), 1:50 (w/w), 1:60 (w/w), 1:70 (w/w), 1:90 (w/w), 1:100 (w/w), or any ratio in between.

In certain embodiments, the plant flour and adhesive has a viscosity of between 6-10,000 centipoise (cP) when mixed. In other embodiments the viscosity of the plant flour mixed with adhesive may be from 10 cP to 8000 cP, from 50 cP to 7000 cP, from 100 cP to 6000 cP, from 200 cP to 5000 cP, from 300 cP to 4000 cP, from 400 cP to 3000 cP, or from 500 cP to 2000 cP. For example, the viscosity of plant flour mixed with adhesive may be about 6 cP, 10 cP, 50 cP, 100 cP, 500 cP, 950 cP, 1000 cP, 2000 cP, 3000 cP, 4000 cP, 5000 cP, 6000 cP, 7000 cP, 8000 cP, 9000 cP, 10000 cP, or any other value in between.

### EXAMPLES

### Example 1

Commercially available strains of cannabis are available in dried form and typically contain in the neighborhood of 20% total actives (THC, CBD and terpenes). Analytical data provided by licensed growers details the ratio of THC, CBD and major terpenes as well as the fraction of total dry matter. Based on analytical data, the proportion of dried milled material to an adhesive formulation may be calculated and after mixing to uniformity the adhesive+active+inert mixture may be incorporated into transdermal delivery devices by extrusion or solvent casting.

Dry-milled cannabis flour (pass 200 mesh) was blended into a polyisobutylene-heptane adhesive mixture to a concentration of 10% cannabis flour by weight and a viscosity between 6-10,000 CPoise. The resultant mixture could be coated on a substrate (backing) and dried down to eliminate the solvent heptane yielding a sticky film on an inert backing material. Optionally, and in the course of a manufacturing operation intended to produce unit dose forms, a release liner can be laminated onto this intermediate product prior to cutting unit dose forms of an appropriate size (depending on desired exposure). Alternately casting can be done on the release liner. When applied to the skin of a subject the mixture of cannabinoids and terpenes will diffuse across the skin and enter the subject's circulation in parallel and in proportion to the chemical potential and diffusion constants of each molecular species in the mixture.

A natural and balanced mixture of active components can thus be presented for transdermal delivery. Flux rates for different components will differ according to the physical-chemical properties of the specific components and thus delivery to into the circulatory system will not necessarily be proportional to the composition of the initial mixture. This restriction is no different from other modalities and data is not available to qualify the differential benefit of different formulations without further testing.

### Example 2

The common spice ‘turmeric’ contains approximately 5% of the pharmacologically active compound curcumin. Curcumin is sparingly water soluble but is lipid soluble and thus suited to transdermal delivery. Turmeric can be incorporated in a suitable transdermal adhesive as in Example 1 and thus presented in a form suitable for direct delivery into the circulation with significantly improved bioavailability and delivery kinetics.

Although the compositions and methods disclosed herein has been described with reference to certain specific embodiments, various modifications thereof will be apparent to those skilled in the art without departing from the spirit and scope of the disclosure. All such modifications as would be apparent to one skilled in the art are intended to be included within the scope of the following claims.

We claim:

1. A pharmaceutical or nutraceutical composition comprising a plant flour mixed with an adhesive.

2. The composition of claim 1, wherein the plant flour comprises one or more plants selected from the group consisting of Cannabis, Curcurma (turmeric), Olea (olive), Theobroma (cocoa), Camellia (tea), Vitis (grape) and Cinnamomum (cinnamon).

3. The composition of claim 1, wherein the plant flour has an average particle diameter of 30 μm-80 μm.

4. The composition of claim 1, wherein the plant flour has a particle diameter that passes through a screen of 80 mesh-400 mesh.

5. The composition of claim 1, wherein the plant flour is mixed with the adhesive at a ratio of plant flour to adhesive from 1:1 (w/w) to 1:100 (w/w).

6. The composition of claim 1, wherein the composition has a viscosity of between 4-1000 cP.

7. The composition of claim 1, wherein the adhesive is selected from the group consisting of an acrylic polymer, a silicone polymer, urethane, isobutylene, polyisobutylene (PIB), vinyl acetate and styrene polymers or copolymers.

8. The composition of claim 1, wherein the plant flour comprises cannabis.

9. The composition of claim 8, wherein the adhesive is selected from the group consisting of acrylic polymer, silicone polymer and PIB.

10. A method of manufacturing a pharmaceutical or nutraceutical composition comprising grinding plant material into a flour and mixing the flour with an adhesive.

11. The method of claim 10, wherein the plant flour comprises one or more plants selected from the group consisting of Cannabis, Curcurma (turmeric), Olea (olive), Theobroma (cocoa), Camellia (tea), Vitis (grape) and Cinnamomum (cinnamon).

12. The method of claim 10, wherein the plant flour has an average particle diameter of 30 μm-80 μm.

13. The method of claim 10, wherein the plant flour has a particle diameter that passes through a screen of 80 mesh-400 mesh.

14. The method of claim 10, wherein the plant flour is mixed with the adhesive at a ratio of plant flour to adhesive from 1:1 (w/w) to 1:100 (w/w).

15. The method of claim 10, wherein the composition has a viscosity of between 4-1000 cP.

16. The method of claim 10, wherein the indicated adhesive is selected from the group consisting of an acrylic polymer, a silicone polymer, urethane, isobutylene, PIB, vinyl acetate and styrene polymers or copolymers.

17. The method of claim 10, wherein the plant flour comprises cannabis.

18. The method of claim 17, wherein the adhesive is selected from the group consisting of acrylic polymer, silicone polymer and PIB.

19. A method of administering tetrahydrocannabinol (THC), cannabidiol (CBD) and/or terpenes to a subject in need thereof comprising mixing a cannabis flour with an adhesive.

20. The method of claim 19, wherein the plant flour has an average particle diameter of 30 μm-80 μm.

21. The method of claim 19, wherein the plant flour has a particle diameter that passes through a screen of 80 mesh-400 mesh.

22. The method of claim 19, wherein the plant flour is mixed with the adhesive at a ratio of plant flour to adhesive from 1:1 (w/w) to 1:100 (w/w).

23. The method of claim 19, wherein the composition has a viscosity of between 4-1000 cP.

24. The method of claim 19, wherein the adhesive is selected from an acrylic polymer, a silicone polymer, urethane, isobutylene, PIB, vinyl acetate and styrene polymers or copolymers.

25. The method of claim 19, wherein the adhesive is selected from the group consisting of acrylic polymer, silicone polymer and PIB.

26. A kit comprising a composition comprising plant flour and an adhesive, wherein the plant flour is mixed with the adhesive at a ratio of plant flour to adhesive from 1:1 (w/w) to 1:100 (w/w), wherein the adhesive is cast with a dry thickness of between about 45 μm and about 95 μm, and wherein the adhesive is laminated with a backing membrane.

27. The kit of claim 26, wherein the plant flour comprises one or more plants selected from the group consisting of Cannabis, Curcurma (turmeric), Olea (olive), Theobroma (cocoa), Camellia (tea), Vitis (grape) and Cinnamomum (cinnamon).

28. The kit of claim 26, wherein the plant flour has an average particle diameter of 30 μm-80 μm.

29. The kit of claim 26, wherein the plant flour has a particle diameter that passes through a screen of 80 mesh-400 mesh.

30. The kit of claim 26, wherein the indicated adhesive is selected from the group consisting of an acrylic polymer, a silicone polymer, urethane, isobutylene, PIB, vinyl acetate and styrene polymers or copolymers.

31. The kit of claim 26, wherein the plant flour comprises cannabis.

32. The kit of claim 31, wherein the adhesive is selected from the group consisting of acrylic polymer, silicone polymer and PIB.

33. The kit of claim 26, wherein the backing membrane is constructed of a material selected from the group consisting of polyesters, polycarbonates, polyimides, polyethylene, poly(ethylene terphthalate), polypropylene, polyurethanes and polyvinylchlorides.
